(19) United States
(12) Patent Application Publication
(10) Pub. No.: US 2026/0125730 A1
(43) Pub. Date: May 7, 2026

(54) A NEW INDUCER FOR METHANOL-FREE PICHIA PASTORIS EXPRESSION SYSTEM

(71) Applicant: ATATURK UNIVERSITESI REKTORLUGU BILIMSEL ARASTIRMA PROJELERI ( BAP ) KOORDINASYON BIRIMI, ERZURUM (TR)

(72) Inventors: YAGMUR UNVER, ERZURUM (TR); MELEK ACAR, ERZURUM (TR); SEYDA YILDIZ, ERZURUM (TR); MESUT TASKIN, ERZURUM (TR)

(73) Assignee: ATATURK UNIVERSITESI REKTORLUGU BILIMSEL ARASTIRMA PROJELERI ( BAP ) KOORDINASYON BIRIMI, ERZURUM (TR)

(21) Appl. No.: 18/695,378

(22) PCT Filed: Mar. 10, 2023
(86) PCT No.: PCT/TR2023/050241

§ 371 Date: Mar. 26, 2024

(30) Foreign Application Priority Data
Mar. 10, 2022 (TR) 2022003632

(51) Int. Cl. C12P 21/00; C12N 15/81

(52) U.S. Cl. CPC C12P 21/00; C12N 15/815; C12N 2800/102; C12N 2830/002

(57) ABSTRACT

The present invention relates to a method for the expression of genes belonging to different organisms (bacteria, fungi, plants, animals and humans) in P. pastoris cells, comprising the following processing steps;
i. Transfer of the gene to be expressed into the plasmid carrying the alcohol oxidase 1 (AOX1) promoter
ii. Cloning of the plasmid carrying the gene
iii. Transfer of the recombinant plasmid carrying the AOX1 promoter and gene into the expression host
iv. Induction of the AOX1 promoter with farnesol and consequent expression of the heterologous gene.

The present invention will be used in the field of recombinant protein production.

### FIELD OF THE INVENTION

The present invention is intended to be used in the field of recombinant protein production wherein the expression of different genes of various microorganisms, plants, animals and humans is performed and thus the production of a large number of different recombinant proteins is carried out.

### STATE OF THE ART

Lately, inducible expression systems have attracted a lot of attention by researchers and many recombinant proteins can be produced with the help of these systems. In this sense, Pichia pastoris (Komagataella phatfii), a methylotrophic yeast (which can use methanol as a carbon and energy source), is one of the most important expression systems used for both intracellular and extracellular recombinant protein production. More than 5000 different proteins have been produced via the P. pastoris expression system in the last 30 years until today, The promoter (P_{AOX1}) of the alcohol oxidase 1 (AOX1) gene, which encodes the alcohol oxidase (AOX) enzyme, is mostly used in the production of high levels of recombinant proteins with this popular expression system. P_{AOX1} is strongly and tightly controlled by means of the methanol induction. Expression of human, plant, animal and microorganism genes is accomplished in P. pastoris by inducing this promoter with methanol. However, it is known that the use of methanol as an inducer has several disadvantages, In particular for the large-scale fermentation industry:
1) It is flammable and toxic.
2) A high oxygen supply is required for its catabolism, and thus this will result in increased production costs due to significant heat release.
3) It is not preferred for producing pharmaceutical or edible products.
4) Its cost will increase in the event of an oil crisis and
5) The by-product of methanol metabolism, hydrogen peroxide (H₂O₂), causes oxidative stress, thus resulting in proteolytic degradation of recombinant proteins. A methanol-free expression system is required so as to eliminate all these problems.

The following patents are encountered which are for obtaining the methanol-free expression system.

EP2873734 B1—Method of Eliminating Dependence of Methanol Induced Promoter on Single Methanol Carbon Source

EP2106447 B1, U.S. Pat. No. 8,236,528B2—Method for Methanol Independent Induction from Methanol Inducible Promoters in Pichia

Although both patents revealed a methanol-free process, they do not contain any description for the use of farnesol.

Furthermore, ‘SNP (sodium nitropurisside)’ was used as an inducer of the AOX promoter for methanol-free P. pastoris expression, and a high level of recombinant protein expression was observed with promoter induction in the patent application titled ‘A method for recombinant protein production’, published on 2022 Jul. 14 (Pub. No.: US 2022/0220491 A1). ‘Farnesol’ is proposed as an inducer of AOX promoter for methanol-free expression of P. pastoris within the scope of this invention.

### DEFINITION OF THE INVENTION

Said invention eliminates the disadvantages described in the state of the art and fulfils the needs.

Said invention is intended to be used in the field of recombinant protein production wherein the expression of different genes of various microorganisms, plants, animals and humans is performed and thus the production of a large number of different recombinant proteins is carried out.

Today, the P. pastoris yeast expression system is used in the production of more than 5000 recombinant proteins, more than 70 of which are therapeutic proteins, available on the market. Although different promoters have been used for protein expression with this system, higher protein production is achieved with alcohol oxidase 1 (AOX1) promoter. The AOX1 promoter (P_{AOX1}) is a methanol-inducible promoter. That is, for the expression of a gene inserted downstream of this promoter, and hence the production of the protein of interest, certain amounts (0.5-4% v/v) and regular intervals (every 12 or 24 hours throughout the cultivation) of methanol must be added to the media of yeast cells. However, the use of methanol as an inducer has numerous disadvantages, as noted above, in particular for the large-scale fermentation industry. The use of methanol was completely eliminated and the protein production was carried out under P_{AOX1} control in recombinant P. pastoris cells with the help of the present invention. That is, a methanol-free expression method was designed for P. pastoris cells. Thus, we propose to eliminate all the disadvantages of methanol and use a less costly inducing agent instead of methanol.

The present invention is to eliminate the disadvantage of using methanol as an inducing agent that allows high amount of protein expression in P. pastoris cells, thus solving the problems it causes in particular in the large-scale fermentation industry.

A methanol-free expression system is proposed in this invention so as to solve the problems caused by said disadvantages of using methanol. Therefore, farnesol has been used instead of methanol as the inducer of the AOX1 promoter in the present invention.

Quorum Sensing Molecules (QSM) are metabolites produced by bacteria or lungi that control responses to internal and external stimuli providing interaction between the cells of these organisms. In microorganisms, QSMs are responsible for regulating ecologically and medically important properties such as bioluminescence, biofilm formation, secretion of virulence factors, sporulation and antibiotic production. Fungal QSMs comprise farnesol, tyrosol, phenylethanol and triptohol.

Farnesol is a 15 carbon natural organic compound with the formula C₁₅H₂O₆. Farnesol, synthesized by plants and fungi, is synthesized from an alcohol called farnesyl pyrophosphate in plant cells. Farnesol functions as a quorum sensing molecule in fungi. Farnesol prevents hyphal morphology by inhibiting germ tube formation in Candida albicans which is a fungus. With the exception of controlling fungal morphology, exogenous farnesol has also been reported to increase the synthesis of enzymes and extracellular polysaccharides in fungi. Farnesol has also been reported to increase prodigiosin synthesis in bacterium Serratia marcescens.

Farnesol can be produced in high quantities by chemical synthesis. It is mentioned that this molecule does not cause toxic effects on humans, and farnesol and its derivatives can even be used as anti-biofilm, anti-cancer, anti-tumor, anti-microbial, anti-inflammatory, anti-allergic and anti-obesity agents in human studies. For this reason, the use of farnesol in the production of recombinant protein will not cause human toxicity.

When the action mechanism of farnesol is examined, it is seen that this molecule changes the expression levels of various genes in microorganisms. This study was carried out so as to test the utility of farnesol instead of methanol as an Inducer in inducing the AOX1 promoter in P. pastoris yeast, to express a heterologous gene inserted in the downstream of the AOX1 promoter and to obtain the protein product of this gene.

Induction of the AOX1 promoter in the present invention was achieved by using ‘farnesol’ instead of methanol. The use of large amount of methanol (at least 5 ml/L) is required during the production of large amount of recombinant protein in an industrial scale. The inducer cost will be reduced by using a very low amount of farnesol (240 μl/L) in the present invention. In addition, the danger of methanol being combustible and taking up too much space during storage will also be eliminated.

Some studies have been found in the literature on recombinant protein production through the Pichia pastoris expression system by eliminating the above-mentioned disadvantages of methanol. The applications included in these studies are given below:
Shen et al. (2016) obtained a new methanol-free expression system by targeting kinases involved in the activation or inhibition of the AOX1 promoter by different carbon sources. Therefore, they obtained two kinase mutants (Δgut1 and Δdak) and reported that the mutants showed strong alcohol oxidase activity without methanol. In conclusion, two different methanol-free expression systems with glycerol-inducible AOX1 promoter (Δgut1-HpGCY1-glycerol) and dihydroxyacetone-inducible AOX1 promoter (Δdak-DHA) were developed. When the two mutant systems were compared, it was seen that the Δdak-DHA system was better. In addition, it has also been reported that the methanol-free expression system of Δdak-DHA is better than the structural GAP promoter and can reach 50-60% of the conventional methanol-induced system.
Wang et al (2017) tested the functions of transacting factors of the AOX1 promoter and regulated them in a combinatorial manner, thus created the methanol-free AOX1 promoter in which three transcriptional repressors (Mig1, Mig2 and Nrgl) were identified and deleted, and a transcription activator (Mit1) was over-expressed, and the starting strain carrying the GFP gene in the downstream of this promoter. When compared with methanol-inducible natural strain, this strain Increased the level of GFP production by 77% in medium containing glycerol, which is a suppressor of the AOX promoter. Then, it became possible to produce insulin precursor (IP) protein in this P. pastoris strain without the use of methanol.
Shirvani et al (2019) developed a methanol-free expression system for the extracellular production of human granulocyte macrophage-colony stimulating factor (hGM-CSF), a pharmaceutical protein playing an important role in the proliferation and differentiation of immune cells. For this purpose, a new expression vector [pEP(α)101] carrying the FMD (formate dehydrogenase) promoter, which regulates heterologous gene expression in the presence of glycerol, was designed. The expression of recombinant hGM-CSF in three different culture media was searched. It has been reported according to the obtained results that the new methanol-free PFMD expression system will be a suitable candidate for heterologous gene expression, in particular for use in food and for the production of therapeutically important recombinant proteins.

As stated in the studies above, during the development of the methanol-free expression systems, the researchers focused on searching for alternative new promoters to the AOX promoter, inhibiting and/or activating existing transcription factors, and creating mutant strains capable of making expression in the absence of methanol.

Such genetic manipulations result in high costs as well as long and laborious processes in production of recombinant proteins via P. pastoris. Furthermore, studies have revealed that developed methanol-free expression systems. generally provide lower yields than methanol-dependent ones. This highlights that the systems tried to be developed do not provide much advantage in terms of product yield. In brief, although methanol-free expression systems, which are currently offered as a solution in the literature, eliminate the use of methanol, they cause the emergence of the above-mentioned new technical problems.

Since our invention proposes a methanol-free expression system, by eliminating the use of methanol;
1. The inducer cost is reduced,
2. The danger of methanol being combustible and taking up too much space during storage is eliminated,
3. Less inducer will be used than methanol.

### BRIEF DESCRIPTION OF DRAWINGS

FIG. 1A.

Western blot analysis result with anti-azurin antibody (M: Marker, 1: Culture broth obtained as a result of Induction of recombinant cells with 0.5% (v/v) methanol, 2, 3 and 4: Culture broths obtained as a result of induction of recombinant cells with 120, 240 and 480 μL/L farnesol, respectively and 5: Culture broth of P. pastoris cells transferred to pPICZαA (control)

FIG. 1B. Relative expression levels of azurin protein in culture broths

### DESCRIPTION OF THE INVENTION

The process steps of our present invention can be summarized as follows:
A. Transfer of the gene to be expressed into the plasmid (pPICZαA) carrying alcohol oxidase (AOX1) promoter
B. Cloning of the plasmid carrying the gene
C. Transfer of the recombinant plasmid carrying the AOX1 promoter and the gene into the expression host (P. pastoris cells)
D. Production of recombinant protein by induction of the AOX1 promoter

Recombinant yeast cells were obtained by performing steps A, B and C in the study in which the recombinant DNA obtained by Inserting the azurin gene of Pseudomonas aeruginosa into pPICZαA was integrated into the genome of P. pastoris X33 strain (https://pubmed.ncbi.nlm.nih.gov/33346686/).

While methanol is frequently used as a promoter inducer in step D, recombinant protein (azurin) production has been performed by using farnesol instead of methanol in the present invention.

In this Invention, recombinant Pichia pastoris cells which carry the “azurin protein” gene (as a reference gene) from Pseudomonas aeruginosa in their genomes and can perform the extracellular production of this protein were used.

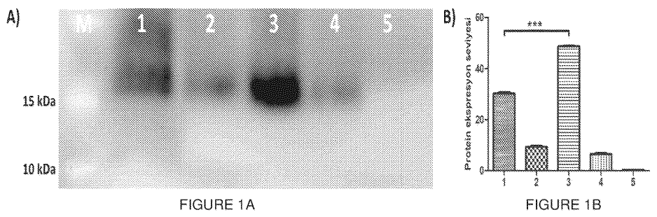

Said protein gene is located in the downstream of the AOX1 promoter in the genome of P. pastoris cells. If the AOX promoter is induced, expression of this gene inserted into the genome can be achieved. Protein expression analyses (western blot) demonstrated that as in the group which methanol (0.5% v/v) was used as a control for the induction of the promoter region, protein production also occurred in groups induced by farnesol at different concentrations (120 μL/L, 240 μL/L and 480 μL/L) Sorbitol, which is known to be non-repressor for the AOX promoter, was used as the carbon source in the production medium in this cultivation process. Methanol or farnesol was added to the culture medium In predetermined amounts every 24 hours. When the cultures were incubated at 30° C. and 280 rpm for 72 hours, it was seen that as in methanol-induction group (in the control group), extracellular protein production was also achieved with the use of farnesol as an inducer (FIG. 1A). Among the farnesol groups, 240 μU/L farnesol group resulted in the highest protein expression level. It was also seen that the protein expression level seen in this group was higher than that of the 0.5% (v/v) methanol-induced group (P<0.05) (FIG. 1B).

1. The invention relates with the discovery of a new inducer for the methanol-free Pichia pastoris expression system. The property of the Invention is the use of farnesol in methanol-inducible promoter induction in the P. pastoris expression system for recombinant protein production.

2. Farnesol, a new inducer for the methanol-free Pichia pastoris expression system according to claim 1, is used for the induction of the methanol-inducible AOX1 promoter in the P. pastoris expression system for recombinant protein production.

3. Farnesol, a new inducer for the methanol-free Pichia pastoris expression system according to claim 1, is used to induce other inducible (AOX2, FLD1 and PEX8) promoters in P. pastoris for recombinant protein production.

4. Farnesol, a new inducer for the methanol-free Pichia pastoris expression system according to claim 1, is used to induce promoters such as CUP1, DD12 and MOX for the production of recombinant protein in other yeast species (such as Saccharomyces cerevisiae, Hansenula polymorpha and Schizosaccharomyces pombe).

5. Farnesol, a new inducer for the methanol-free Pichia pastoris expression system according to claim 1, is used as an inducer for recombinant protein production in all bacteria (such as Escherichia coli, Bacillus subtilis and Lactococcus lactis), molds (such as Aspergillus niger, A. oryzae and Trichoderma reesei), macrofungi and other organisms (such as algae).

6. Farnesol, a new inducer for the methanol-free Pichia pastoris expression system according to claim 1, is used as an Inducer for recombinant protein production in all mammalian (such as Chinese Hamster Ovary; CHO cells) and insect expression systems.

7. Farnesol precursor molecules such as mevalonic acid, geranyl pyrophosphate (GPP) and farnesyl diphosphate and farnesol conversion products such as farnesol and farnesoic acid according to claim 1, are also used as inducers for recombinant protein production via yeast, bacteria, molds and algae.

8. Farnesol derivatives such as farnesyl acetate, α-farnesene, β-farnesene and nerolidol according to claim 1 are also used as inducers for recombinant protein production via yeast, bacteria, molds and algae.

9. Fungal quorum sensing molecules such as tryptofol, tyrosol, multicholic acid, multicholanic acid and phenylethanol, α-(1,3)-glucan, γ-heptalactone and butyrolactone I according to claim 1 are used as inducers for recombinant protein production via yeast, bacteria, molds and algae.

10. The use of bacterial quorum sensing molecules (autoinducers) according to claim 1 are used as inducers for recombinant protein production via yeast. bacteria, molds and algae.

11. Method of using a new inducer for the methanol-free Pichia pastoris expression system, characterized in that, it comprises the following process steps:
i. Transfer of the gene to be expressed into the plasmid carrying the alcohol oxidase 1 (AOX1) promoter
ii. Cloning of the plasmid carrying the gene
iii. Transfer of the recombinant plasmid carrying the AOX1 promoter and gene into the expression host
iv. Induction of the AOX1 promoter with farnesol and consequent expression of the heterologous gene.
